(19) United States
(12) Patent Application Publication
(10) Pub. No.: US 2022/0227568 A1
(43) Pub. Date: Jul. 21, 2022

(54) Glove Dispenser Assembly

(71) Applicant: Fania Brunache, Lake Worth, FL (US)

(72) Inventor: Fania Brunache, Lake Worth, FL (US)

(21) Appl. No.: 17/151,797

(22) Filed: Jan. 19, 2021

(51) Int. Cl. B65D 83/08; A41D 19/015

(52) U.S. Cl. CPC B65D 83/0894; A61B 42/40; A41D 19/015

(57) ABSTRACT

A glove dispenser assembly includes a housing that is mounted to a vertical support surface and the housing has a discharge chute integrated therein. The housing has a plurality of compartments therein to contain a respective box of sanitary gloves. A plurality of dispensing units is included and each of the dispensing units is positioned within the housing. Each of the dispensing units is positioned beneath a respective one of the compartments to engage a glove in the box of sanitary gloves that is positioned in the respective compartment. A plurality of motion sensors is each coupled to the housing wherein each of the motion sensors is configured to sense motion proximate the housing and a respective dispensing unit is turned on when the motion sensor associated with the respective dispensing unit senses motion.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to dispenser devices and more particularly pertains to a new dispenser device for automatically dispensing a sanitary glove. The dispenser device includes a plurality of motion sensors and the dispenser device dispenses one single glove at a time. Additionally, the dispenser device stores a variety of sizes of sanitary gloves and the dispenser device can dispense a selected size of sanitary gloves.

### (2) Description of Related Art Including Information Disclosed Under 37 CFR 1.97 and 1.98

The prior art relates to dispenser devices including a plurality of storage devices for storing sanitary gloves in a manner that facilitates a single sanitary glove to be removed. The prior art discloses a variety of dispensing units that include a roller around which a plurality of sanitary gloves are wrapped and which can dispense the sanitary gloves from the roller.

### BRIEF SUMMARY OF THE INVENTION

An embodiment of the disclosure meets the needs presented above by generally comprising a housing that is mounted to a vertical support surface and the housing has a discharge chute integrated therein. The housing has a plurality of compartments therein to contain a respective box of sanitary gloves. A plurality of dispensing units is included and each of the dispensing units is positioned within the housing. Each of the dispensing units is positioned beneath a respective one of the compartments to engage a glove in the box of sanitary gloves that is positioned in the respective compartment. A plurality of motion sensors is each coupled to the housing wherein each of the motion sensors is configured to sense motion proximate the housing and a respective dispensing unit is turned on when the motion sensor associated with the respective dispensing unit senses motion.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a glove dispenser assembly according to an embodiment of the disclosure.

FIG. 2 is a front view of an embodiment of the disclosure.

FIG. 3 is a back phantom view of an embodiment of the disclosure.

FIG. 4 is a right side phantom view of an embodiment of the disclosure.

FIG. 5 is a perspective view of an alternative embodiment of the disclosure.

### DETAILED DESCRIPTION OF THE INVENTION

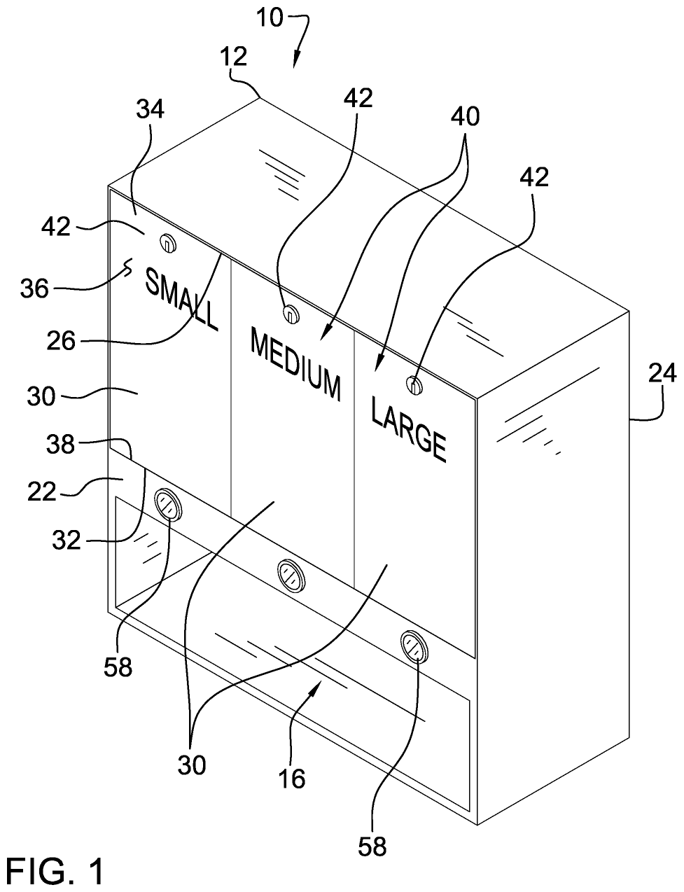

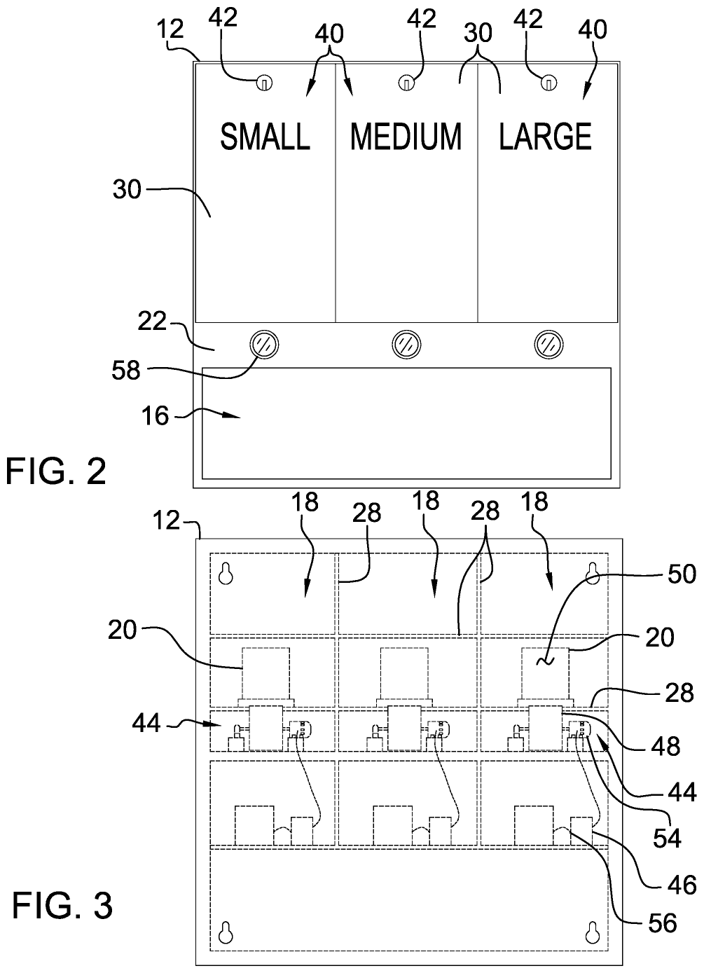

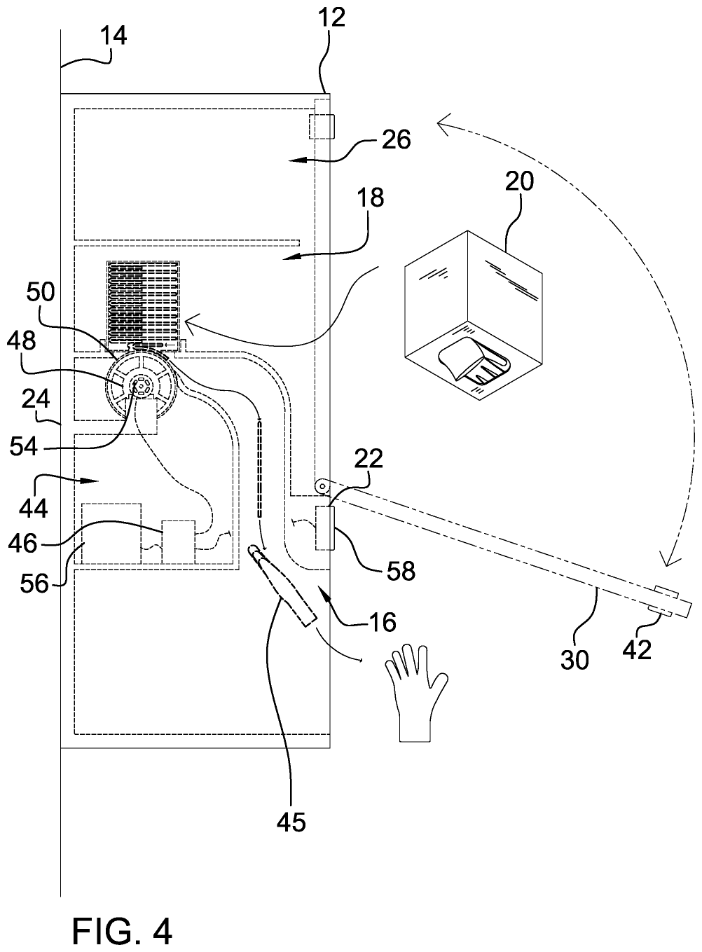

With reference now to the drawings, and in particular to FIGS. 1 through 5 thereof, a new dispenser device embodying the principles and concepts of an embodiment of the disclosure and generally designated by the reference numeral 10 will be described.

As best illustrated in FIGS. 1 through 5, the glove dispenser assembly 10 generally comprises a housing 12 that is mounted to a vertical support surface 14, such as a wall or the like. In this way the housing 12 is accessible to a user having the housing 12 being positioned at approximately eye level for the user. The wall may be an interior wall of clinic, hospital, or other setting that commonly employs sanitary practices to reduce the transmission of infectious diseases. The housing 12 has a discharge chute 16 that is integrated therein and the housing 12 has a plurality of compartments 18 therein. Each of the compartments 18 contains a respective box of sanitary gloves 20 that each has a unique glove size with respect to each other. Additionally, the discharge chute 16 is in communication with each of the compartments 18.

The housing 12 has a front wall 22 and a back wall 24, and the discharge chute 16 extends through the front wall 22 into an interior of the housing 12. Moreover, the discharge 16 chute extends across a full width of the front wall 22. The front wall 22 has an access opening 26 extending into the interior of the housing 12 and the access opening 26 extends across a full width of the front wall 22. The housing 12 has a plurality of dividers 28 therein to define the plurality of compartments 18 within the housing 12. The dividers 28 are spaced apart from each other and are distributed along the front wall 22 such that each of the compartments 18 is accessible through the access opening 26.

A plurality of doors 30 is provided and each of the doors 30 is hingedly coupled to the housing 12. Each of the doors 30 is aligned with a respective one of the compartments 18 and each of the doors 30 covers the respective compartment 18 when the doors 30 are closed. Each of the doors 30 exposes the respective compartment 18 when the doors 30 are opened, and the doors 30 are independently closable or openable. Each of the doors 30 has a lower edge 32, an upper edge 34 and a front surface 36. The lower edge 32 of each of the doors 30 is hingedly coupled to a lower bounding edge 38 of the access opening 26. The front surface 36 of each of the doors 30 has indicia 40 printed thereon, and the indicia 40 on respective ones of the doors 30 comprises the words “small”, “medium” and “large”.

A plurality of locks 42 is provided and each of the locks 42 is coupled to a respective one of the doors 30. Each of the locks 42 is positioned adjacent to the upper edge 34 of the respective door. Additionally, each of the locks 42 engages the front wall 22 of the housing 12 when said locks 42 are in a locked position and the respective door is closed. In this way the doors 30 are locked closed and can only be opened by an authorized person. Each of the locks 42 may comprise a keyed lock, a combination lock or other type of mechanical lock.

A plurality of dispensing units 44 is provided and each of the dispensing units 44 is positioned within the housing 12. Each of the dispensing units 44 is positioned beneath a respective one of the compartments 18. In this way each of the dispensing units 44 can engage a glove 45 in the box of sanitary gloves 20 that is positioned in the respective compartment 18. Each of the dispensing units 44 completes one dispensing cycle each time the dispensing units 44 is turned on to dispense one glove 45 at a time.

Each of the dispensing units 44 comprises a control circuit 46 that is integrated into the housing 12, and the control circuit 46 receives a dispense input. Each of the dispensing units 44 includes a wheel 48 has an outer surface 50. The wheel 48 is rotatably positioned within the housing 12 and the wheel 48 has a rotational axis that is oriented parallel to the front wall 22 of the housing 12. The wheel 48 is positioned at a junction between the discharge chute 16 and a respective one of the compartments 18. In this way the outer surface 50 of the wheel 48 can frictionally engage a glove 45 in the box of sanitary gloves 20 that is positioned in the respective compartment 18. Moreover, the outer surface 50 of the wheel 48 is comprised of a friction enhancing material, including but not being limited to, rubber or silicone, thereby facilitating the outer surface 50 to enhance frictionally engaging the glove 45.

Each of the dispensing units 44 includes a motor 54 that is positioned within the housing 12. The motor 54 is electrically coupled to the control circuit 46 and the motor 54 is rotatably coupled to the wheel 48. The motor 54 rotates in a first direction for a predetermined number of rotations when the motor 54 is turned on such that the wheel 48 is rotated a predetermined number of rotations when the motor 54 is turned on. In this way the wheel 48 urges a single glove from the box of sanitary gloves 20 each time the motor 54 is turned on thereby facilitating the single glove to fall downwardly into the discharge chute 16 for retrieval. Additionally, the motor 54 is turned on when the control circuit 46 receives the dispense input. The motor 54 may comprise an electric motor or the like. Each of the dispensing units 44 includes a power supply 56 that is positioned in the housing 12. The power supply 56 is electrically coupled to the control circuit 46 and the power supply 56 comprises at least one battery.

A plurality of motions sensors 58 is provided and each of the motions sensors 58 is coupled to the housing 12 to sense motion proximate the housing 12. Each of the motions sensors 58 is in communication with a respective one of the dispensing units 44. Moreover, the respective dispensing unit 44 is turned on when the motion sensor 58 associated with the respective dispensing unit 44 senses motion. In this way the dispensing units 44 can dispense a glove 45 of a selected size when a user waves their hand in front of the motion sensor 58 that is associated with the respective dispensing unit 44. Each of the motions sensors 58 is electrically coupled to the control circuit 46 of the respective dispensing unit 44. The control circuit 46 in the respective dispensing unit 44 receives the dispense input when the motion sensor 58 associated with the respective dispensing unit 44 senses motion. Each of the motions sensors 58 may comprise an electronic motion sensor or the like, including but not being limited to, an infra-red motion sensor. Each of the motion sensors 58 is positioned on the front wall 22 of the housing 12 between the plurality of doors 30 and the discharge chute 16.

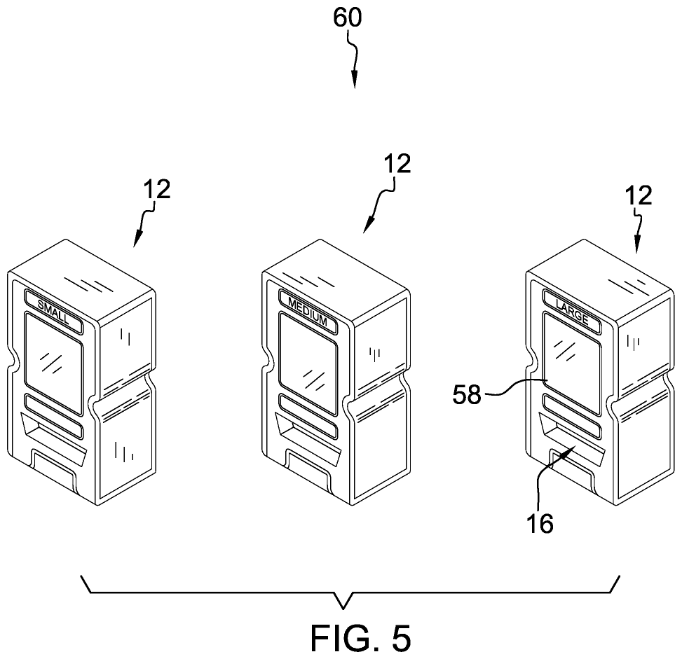

In an alternative embodiment 60 as is most clearly shown in FIG. 5, a plurality of housings 12 is provided and each of the housings 12 has a discharge chute 16 integrated therein. Additionally, each of the housings 12 contains a respective box of sanitary gloves 20. Each of the dispensing units 44 is positioned in a respective one of the housings 12 to dispense a glove 45 of a respective size from the housing 12. In this way the user can approach the housing 12 in which their desired size of box of sanitary gloves 20 is contained.

In use, the user approaches the housing 12 and the user waves their hand in front of the motion sensor that is associated with the size of glove the user wishes to dispense. Thus, the dispenser unit associated with the motions sensor 58 that senses motion is turned on to dispense a single glove 45. In this way the glove 45 can be dispensed in a touch-less manner, thereby reducing transmission of infectious diseases between users. The glove 45 is dropped downwardly into the discharge chute 16 for retrieval. The user can wave their hand in front of the chosen motion sensor as many times as needed to dispense as many gloves 45 as is desired. A respective door 30 is opened to facilitate the box of gloves 20 in a respective compartment 18 to be replaced when the box of gloves 20 is depleted.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A glove dispenser assembly for dispensing a single, sanitary glove for enhancing sterility of a person's hands, said assembly comprising:
a housing being mounted to a vertical support surface wherein said housing is configured to be accessible to a user having said housing being positioned at approximately eye level for the user, said housing having a discharge chute being integrated therein, said housing having a plurality of compartments therein wherein each of said compartments is configured to contain a respective box of sanitary gloves each having a unique glove size with respect to each other, said discharge chute being in communication with each of said compartments;
a plurality of doors, each of said doors being hingedly coupled to said housing, each of said doors being aligned with a respective one of said compartments, each of said doors covering said respective compartment when said doors are closed, each of said doors exposing said respective compartment when said doors are opened, said doors being independently closable or openable;
a plurality of dispensing units, each of said dispensing units being positioned within said housing, each of said dispensing units being positioned beneath a respective one of said compartments wherein each of said dispensing units is configured to engage a glove in the box of sanitary gloves that is positioned in said respective compartment, each of said dispensing units completing one dispensing cycle each time said dispensing units is turned on wherein each of said dispensing units is configured to dispense one sanitary glove at a time; and
a plurality of motion sensors, each of said motion sensors being coupled to said housing wherein each of said motion sensors is configured to sense motion proximate said housing, each of said motion sensors being in communication with a respective one of said dispensing units, said respective dispensing unit being turned on when said motion sensor associated with said respective dispensing unit senses motion wherein said dispensing units is configured to dispense a glove of a selected size when a user waves their hand in front of said motion sensor being associated with said respective dispensing unit.

2. The assembly according to claim 1, wherein said housing has a front wall and a back wall, said discharge chute extending through said front wall into an interior of said housing, said chute extending across a full width of said front wall, said front wall having an access opening extending into said interior of said housing, said access opening extending across a full width of said front wall.

3. The assembly according to claim 3, wherein said housing has a plurality of dividers therein to define said plurality of compartments within said housing, said dividers being spaced apart from each other and being distributed along said front wall such that each of said compartments is accessible through said access opening.

4. The assembly according to claim 2, wherein each of said doors has a lower edge, an upper edge and a front surface, said lower edge of each of said doors being hingedly coupled to a lower bounding edge of said access opening, said front surface of each of said doors having indicia being printed thereon, said indicia on respective ones of said doors comprising the words “small”, “medium” and “large”.

5. The assembly according to claim 2, wherein each of said dispensing units comprises a wheel having an outer surface, said wheel being rotatably positioned within said housing, said wheel having a rotational axis being oriented parallel to said front wall of said housing, said wheel being positioned at a junction between said dispensing chute and a respective one of said compartments wherein said outer surface of said wheel is configured to frictionally engage a glove in the box of sanitary gloves that is positioned in said respective compartment.

6. The assembly according to claim 1, wherein said outer surface of said wheel is comprised of a friction enhancing material wherein said outer surface is configured to enhance frictionally engaging the glove.

7. The assembly according to claim 1, wherein each of said dispensing units includes:
a control circuit being integrated into said housing, said control circuit receiving a dispense input; and
a motor being positioned within said housing, said motor being electrically coupled to said control circuit, said motor being rotatably coupled to said wheel, said motor rotating in a first direction for a predetermined number of rotations when said motor is turned on such that said wheel is rotated a predetermined number of rotations when said motor is turned on wherein said wheel is configured to urge a single glove from the box of sanitary gloves each time said motor is turned on thereby facilitating the single glove to fall downwardly into said dispensing chute for retrieval, said motor being turned on when said control circuit receives said dispense input.

8. The assembly according to claim 7, wherein each of said dispensing units includes a power supply being positioned in said housing, said power supply being electrically coupled to said control circuit, said power supply comprising at least one battery.

9. The assembly according to claim 7, wherein each of said motion sensors is electrically coupled to said control circuit of said respective dispensing unit, said control circuit in said respective dispensing unit receiving said dispense input when said motion sensor associated with said respective dispensing unit senses motion.

10. A glove dispenser assembly for dispensing a single, sanitary glove for enhancing sterility of a person's hands, said assembly comprising:
a housing being mounted to a vertical support surface wherein said housing is configured to be accessible to a user having said housing being positioned at approximately eye level for the user, said housing having a discharge chute being integrated therein, said housing having a plurality of compartments therein wherein each of said compartments is configured to contain a respective box of sanitary gloves each having a unique glove size with respect to each other, said discharge chute being in communication with each of said compartments, said housing having a front wall and a back wall, said discharge chute extending through said front wall into an interior of said housing, said chute extending across a full width of said front wall, said front wall having an access opening extending into said interior of said housing, said access opening extending across a full width of said front wall, said housing having a plurality of dividers therein to define said plurality of compartments within said housing, said dividers being spaced apart from each other and being distributed along said front wall such that each of said compartments is accessible through said access opening;
a plurality of doors, each of said doors being hingedly coupled to said housing, each of said doors being aligned with a respective one of said compartments, each of said doors covering said respective compartment when said doors are closed, each of said doors exposing said respective compartment when said doors are opened, said doors being independently closable or openable, each of said doors having a lower edge, an upper edge and a front surface, said lower edge of each of said doors being hingedly coupled to a lower bounding edge of said access opening, said front surface of each of said doors having indicia being printed thereon, said indicia on respective ones of said doors comprising the words “small”, “medium” and “large”;
a plurality of dispensing units, each of said dispensing units being positioned within said housing, each of said dispensing units being positioned beneath a respective one of said compartments wherein each of said dispensing units is configured to engage a glove in the box of sanitary gloves that is positioned in said respective compartment, each of said dispensing units completing one dispensing cycle each time said dispensing units is turned on wherein each of said dispensing units is configured to dispense one sanitary glove at a time, each of said dispensing units comprising:
a control circuit being integrated into said housing, said control circuit receiving a dispense input;
a wheel having an outer surface, said wheel being rotatably positioned within said housing, said wheel having a rotational axis being oriented parallel to said front wall of said housing, said wheel being positioned at a junction between said dispensing chute and a respective one of said compartments wherein said outer surface of said wheel is configured to frictionally engage a glove in the box of sanitary gloves that is positioned in said respective compartment, said outer surface of said wheel being comprised of a friction enhancing material wherein said outer surface is configured to enhance frictionally engaging the glove;
a motor being positioned within said housing, said motor being electrically coupled to said control circuit, said motor being rotatably coupled to said wheel, said motor rotating in a first direction for a predetermined number of rotations when said motor is turned on such that said wheel is rotated a predetermined number of rotations when said motor is turned on wherein said wheel is configured to urge a single glove from the box of sanitary gloves each time said motor is turned on thereby facilitating the single glove to fall downwardly into said dispensing chute for retrieval, said motor being turned on when said control circuit receives said dispense input; and
a power supply being positioned in said housing, said power supply being electrically coupled to said control circuit, said power supply comprising at least one battery; and
a plurality of motion sensors, each of said motion sensors being coupled to said housing wherein each of said motion sensors is configured to sense motion proximate said housing, each of said motion sensors being in communication with a respective one of said dispensing units, said respective dispensing unit being turned on when said motion sensor associated with said respective dispensing unit senses motion wherein said dispensing units is configured to dispense a glove of a selected size when a user waves their hand in front of said motion sensor being associated with said respective dispensing unit, each of said motion sensors being electrically coupled to said control circuit of said respective dispensing unit, said control circuit in said respective dispensing unit receiving said dispense input when said motion sensor associated with said respective dispensing unit senses motion.

11. The assembly according to claim 10, further comprising a plurality of housings, each of said housings having a dispensing chute integrated therein wherein each of said housings is configured to contain a respective box of sanitary gloves, each of said dispensing units being positioned in a respective one of said housings wherein each of said dispensing units is configured to dispense a glove of a respective size from said housing.
